(19) United States
(12) Patent Application Publication
(10) Pub. No.: US 2025/0100909 A1
(43) Pub. Date: Mar. 27, 2025

(54) USE OF MESH ELECTRODES IN ELECTROLYTIC - CATION EXCHANGE MODULES

(71) Applicant: Evoqua Water Technologies LLC, Pittsburgh, PA (US)

(72) Inventors: Li-Shiang LIANG, Harvard, MA (US); Alex B FILIP, Burlington, MA (US)

(73) Assignee: Evoqua Water Technologies LLC, Pittsburgh, PA (US)

(21) Appl. No.: 18/895,642

(22) Filed: Sep. 25, 2024

Related U.S. Application Data
(60) Provisional application No. 63/540,190, filed on Sep. 25, 2023.

(51) Int. Cl. C02F 1/461; C02F 103/08; C07C 1/12; C25B 1/04; C25B 9/21; C25B 9/75; C25B 9/77; C25B 11/031; C25B 11/052; C25B 11/063; C25B 11/075; C25B 11/081

(52) U.S. Cl. CPC C02F 1/46109; C02F 1/4618; C07C 1/12; C25B 1/04; C25B 9/21; C25B 9/75; C25B 9/77; C25B 11/031; C25B 11/052; C25B 11/063; C25B 11/075; C25B 11/081; C02F 2001/46128; C02F 2001/46138; C02F 2001/46161; C02F 2001/46185; C02F 2103/08; C02F 2201/46115; C02F 2201/46145; C02F 2201/4618

(57) ABSTRACT

An electrochemical system includes first and second endplates and a plurality of electrochemical units disposed between the first and second endplates. Each of the plurality of electrochemical units includes a porous anode disposed in an anode compartment, a porous cathode disposed in a cathode compartment, and a center compartment disposed between and separated from the anode compartment and the cathode compartment by cation exchange membranes.

### CROSS-REFERENCE TO RELATED APPLICATIONS

The present application claim priority to U.S. Provisional Application No. 63/540,190, filed Sep. 25, 2023 and titled “Use of Mesh Electrodes in Electrolytic-Cation Exchange Module”, the disclosure of which is incorporated herein in its entirety.

### BACKGROUND

### 1. Field of Invention

Aspects and embodiments disclosed herein are generally directed to electrochemical devices, and more specifically, to electrolytic-cation exchange modules and methods of fabricating and operating same.

### 2. Discussion of Related Art

Electrochemical devices that perform processes based on chemical reactions at electrodes are widely used in industrial and municipal implementations. Examples of reactions include:
A. Electro chlorination with generation of sodium hypochlorite from sodium chloride and water.
   Reaction at anode: 2Cl⁻ →Cl₂+2e⁻
   Reaction at cathode: 2Na⁺+2H₂O+2e⁻→2NaOH+H₂
   In solution: Cl₂+2OH⁻→ClO⁻+Cl⁻+H₂O
   Overall reaction: NaCl+H₂O→NaOCl+H₂
B. Generation of sodium hydroxide and chlorine from sodium chloride and water, with a cation exchange membrane separating the anode and the cathode:
   Reaction at anode: 2Cl^{-→Cl}₂+2e⁻
   Reaction at cathode: 2H₂O+2e⁻2OH⁻+H₂
   Overall reaction: 2NaCl+2H₂O→2NaOH+Cl₂+H₂

### SUMMARY

In accordance with one aspect, there is provided an electrochemical system. The electrochemical system comprises first and second endplates and a plurality of electrochemical units disposed between the first and second endplates. Each of the plurality of electrochemical units include a porous anode disposed in an anode compartment, a porous cathode disposed in a cathode compartment, and a center compartment disposed between and separated from the anode compartment and the cathode compartment by cation exchange membranes.

In some embodiments, at least one anode compartment functions as an anode compartment for adjacent electrochemical units.

In some embodiments, at least one cathode compartment functions as a cathode compartment for adjacent electrochemical units.

In some embodiments, the porous anode of each of the plurality of electrochemical units is formed of titanium coated with one of ruthenium oxide, iridium oxide, or a mixed metal oxide.

In some embodiments, the porous cathode of each of the plurality of electrochemical units is formed of titanium coated with platinum.

In some embodiments, the system further comprises an electrical power supply, the porous anodes of each of the plurality of electrochemical units electrically coupled in parallel to the power supply, the porous cathodes of each of the plurality of electrochemical units electrically coupled in parallel to the power supply.

In some embodiments, the porous anodes of each of the plurality of electrochemical units are one of mesh electrodes, perforated sheets, or sintered porous sheets.

In some embodiments, the porous cathodes of each of the plurality of electrochemical units are one of mesh electrodes, perforated sheets, or sintered porous sheets.

In some embodiments, the anode compartments further comprise inert porous screens.

In some embodiments, the inert porous screens are disposed between the anodes and adjacent cation exchange membranes.

In some embodiments, the cathode compartments further comprise inert porous screens.

In some embodiments, the inert porous screens are disposed between the cathodes and adjacent cation exchange membranes.

In some embodiments, the system further comprises a source of electrolyte solution fluidly connectable to the anode compartment and the cathode compartment of each of the plurality of electrochemical units.

In some embodiments, the system further comprises a bipolar electrode.

In some embodiments, the bipolar electrode is disposed between a first subset of the plurality of electrochemical units and a second subset of the plurality of electrochemical units.

In some embodiments, the bipolar electrode separates an anode compartment of the first subset of the plurality of electrochemical units from a cathode compartment of the second subset of the plurality of electrochemical units.

In some embodiments, the anode and cathode compartments have widths of about 10 mm or less.

In some embodiments, the system further comprises a power supply electrically coupled to the anodes and cathodes of each of the plurality of electrochemical units and configured to supply sufficient current across the anodes and cathodes of each of the plurality of electrochemical units to acidify a feed stream in the center compartments.

In some embodiments, the system further comprises a source of seawater fluidly connectable to the center compartments of each of the plurality of electrochemical units.

In some embodiments, the system further comprises a power supply electrically coupled to the anodes and cathodes of each of the plurality of electrochemical units and configured to supply sufficient current across the anodes and cathodes of each of the plurality of electrochemical units to cause a pH of the seawater in the center compartment to change to a level at which carbon dioxide is formed from bicarbonate in the seawater.

In some embodiments, the system further comprises a flow controller operable to control a flow rate of the seawater through the center compartments.

In accordance with another aspect, there is provided an electrochemical acidification device. The device comprises first and second endplates and an electrochemical unit disposed between the first and second endplates. The electrochemical unit includes a porous anode disposed in an anode compartment, a porous cathode disposed in a cathode compartment, and a center compartment disposed between and separated from the anode compartment and the cathode compartment by cation exchange membranes.

In some embodiments, the device further comprises a power supply electrically coupled to the anode and cathode and configured to supply sufficient current across the anode and cathode to acidify an aqueous solution in the center compartment and generate hydrogen gas from catholyte in the cathode compartment.

In some embodiments, the device further comprises at least one additional electrochemical unit disposed between the first and second endplates and including at least one additional porous anode disposed in at least one additional anode compartment, at least one additional porous cathode disposed in at least one additional cathode compartment, and at least one additional center compartment disposed between and separated from the at least one additional anode compartment and the at least one additional cathode compartment by cation exchange membranes.

In accordance with another aspect, there is provided a method of acidifying a feed stream. The method comprises providing an electrochemical system including first and second endplates and an electrochemical unit disposed between the first and second endplates, the electrochemical unit including a porous anode disposed in an anode compartment, a porous cathode disposed in a cathode compartment, and a center compartment disposed between and separated from the anode compartment and the cathode compartment by cation exchange membranes, flowing an electrolyte through the anode and cathode compartments, flowing the feed stream through the center compartment, and applying electrical current across the porous anode and porous cathode.

In some embodiments, the method further comprises reacting products generated in the center compartment and cathode compartment to form a fuel.

In some embodiments, the electrolyte is an aqueous solution.

In some embodiments, the method further comprises generating hydrogen ions in the anode compartment and transporting the hydrogen ions into the center compartment.

In some embodiments, the feed stream includes seawater.

In some embodiments, applying the electrical current across the porous anode and the porous cathode includes applying sufficient current across the porous anode and porous cathode to cause a pH of the seawater in the center compartment to change from about 8 to below 4.

In some embodiments, the method further comprises withdrawing hydrogen from the cathode compartment.

In some embodiments, the method further comprises withdrawing carbon dioxide from the center compartment.

In some embodiments, the electrochemical system includes at least one additional electrochemical unit disposed between the first and second endplates and including at least one additional porous anode disposed in at least one additional anode compartment, at least one additional porous cathode disposed in at least one additional cathode compartment, and at least one additional center compartment disposed between and separated from the at least one additional anode compartment and the at least one additional cathode compartment by cation exchange membranes, and applying the electrical current from each pair of porous anodes to the porous cathodes in between the porous anodes.

In some embodiments, applying the electrical current across the porous anode and porous cathode of the electrochemical unit and the at least one additional porous anode and porous cathode of the at least one additional electrochemical unit includes electrically connecting the porous anode and at least one additional porous anode.

In some embodiments, applying the electrical current across the porous anode and porous cathode of the electrochemical unit and the at least one additional porous anode and porous cathode of the at least one additional electrochemical unit includes electrically connecting the porous cathode and to the at least one additional porous cathode.

In some embodiments, applying the electrical current across the porous anode and porous cathode of the electrochemical unit and the at least one additional porous anode and porous cathode of the at least one additional electrochemical unit includes applying a higher voltage to the porous anodes than the porous cathodes.

In some embodiments, the method further comprises applying electrical current to a bipolar electrode disposed between the electrochemical unit and the at least one additional electrochemical unit.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic of an embodiment of an electrolytic-cation exchange module (E-CEM) device and process;

FIG. 2 shows an equilibrium diagram for inorganic carbonic species in seawater at 10° C.;

FIG. 3 illustrates an E-CEM system including multiple modules installed and operated in parallel;

FIG. 4 shows a schematic of an E-CEM system with electrodes in multiple electrochemical units connected back-to-back;

FIG. 5 shows a schematic of an E-CEM system with bipolar electrodes;

FIG. 6 shows a schematic of an electrochemical system with porous electrodes and a plurality of electrochemical units;

FIG. 7 shows a portion of an example of a mesh electrode with a connection welded to one corner;

FIG. 8 shows a hybrid E-CEM system with porous electrodes and bipolar electrodes;

FIG. 9 shows a hypothetical example of a process performed by the system of FIG. 8;

FIG. 10 illustrates a control system that may be utilized for embodiments of electrochemical systems disclosed herein;

FIG. 11 illustrates a memory system for the control system of FIG. 10;

FIG. 12 shows a graph of seawater effluent pH over time in accordance with an embodiment of the present disclosure;

FIG. 13 shows a graph of voltage applied to E-CEM modules over time in accordance with an embodiment of the present disclosure; and

FIG. 14 shows a graph of instantaneous power consumption of E-CEM modules over time in accordance with an embodiment of the present disclosure.

### DETAILED DESCRIPTION

Aspects and embodiments disclosed herein are not limited to the details of construction and the arrangement of components set forth in the following description or illustrated in the drawings. Aspects and embodiments disclosed herein are capable of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

Aspects and embodiments disclosed herein include electrolytic-cation exchange (E-CEM) modules and systems using porous electrodes to separate center compartments and methods of using same. Aspects and embodiments disclosed herein enable multiple electrochemical units to be stacked between a set of closing mechanisms thereby increasing flow rate for an individual system and reducing capital cost, weight and footprint.

The E-CEM process is an electrochemical process that may be used to reduce seawater pH and convert dissolved bicarbonate ions to CO₂ gas while simultaneously producing H₂ gas through electrolytic dissociation of water.

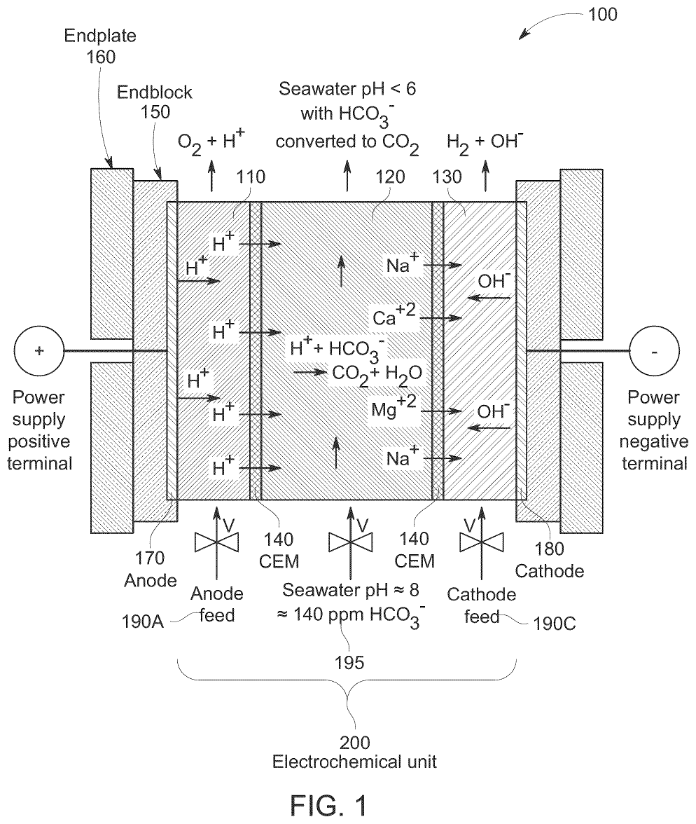

FIG. 1 is a schematic of an embodiment of an E-CEM device and process. The E-CEM device 100 includes three compartments: an anode compartment 110, a center compartment 120, and a cathode compartment 130, separated by cation exchange membranes (CEM) 140. An anode 170 is disposed in the anode compartment 110 opposite one CEM 140 with the anode 170 and CEM 140 defining widthwise boundaries of the anode compartment 110. A cathode 180 is disposed in the cathode compartment 130 opposite another CEM 140 with the cathode 180 and CEM 140 defining widthwise boundaries of the cathode compartment 130. A source of electrolyte solution (feed 190A) is fluidly connectable to the anode compartment 110 and a source of electrolyte solution (feed 190C), which may be the same or different from the electrolyte solution for the anode compartment 110 is fluidly connectable to the cathode compartment 130 of the embodiment shown in FIG. 1 as well as the plurality of anode and cathode compartments 110, 130 of other embodiments discussed herein. The feed 190A, 190C to the anode and cathode compartments 110, 130 is a conductive solution, for example, reverse osmosis (RO) product water with a conductivity <200 μS/cm or sodium sulfate solution with a conductivity of between 300 μS/cm and 3000 μS/cm. With an applied DC current, H⁺ ions generated at the anode 170 are transported through a CEM 140 into the center compartment 120. A source of seawater (feed 195) is fluidly connectable to the center compartment 120 of the embodiment shown in FIG. 1 as well as the plurality of center compartments 120 of other embodiments discussed herein. Seawater feed 195 to the center compartment is acidified and bicarbonate ions in the seawater are converted to carbonous acid (H₂CO₂). Cations in the seawater are transported through the second CEM 140 into the cathode compartment. OH⁻ ions and H₂ (g) are generated at the cathode. Flow controllers, for example, valves V may be disposed on supply lines of any of the feed 190A, 190C to the anode and cathode compartments 110, 130 and/or the seawater feed 195. Such flow controllers may be controlled by a computerized control system as discussed below and may be present in any of the embodiments disclosed herein.

The reactions in the compartments are:
Anode: 2H₂O→4H⁺+O₂(g)+4e⁻
Center: H⁺+HCO₃^{-→H}₂CO₃→H₂O+CO₂(g)
Cathode: 2H₂O+4e⁻→2 OH⁻+2 H₂(g)

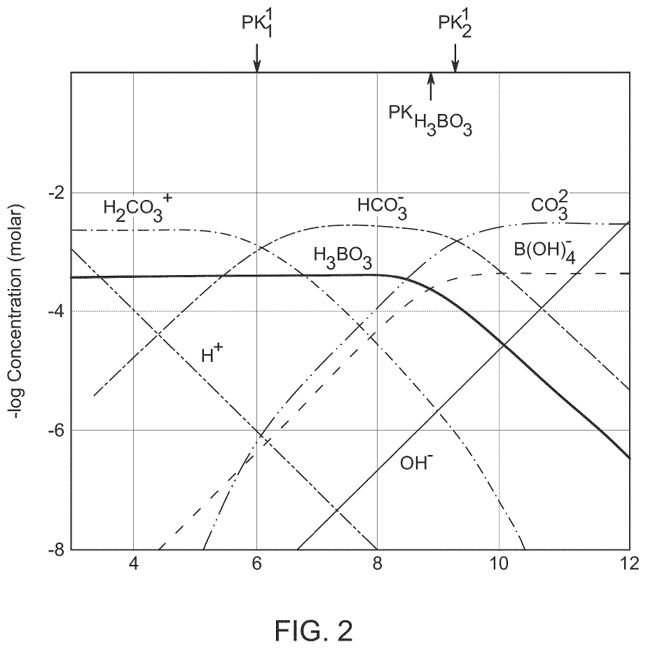

Seawater has a pH of about 8. FIG. 2 shows the equilibrium diagram for inorganic carbonic species in seawater at 10° C. If the pH is reduced to less than about 4, carbonate and bicarbonate in the seawater are converted to H₂CO₃.

CO₂ gas can be withdrawn from the center compartment effluent by vacuum or by membrane degasification. H₂ gas can be withdrawn from the cathode compartment effluent by vacuum or by membrane degasification. The CO₂ and H₂ gases can be used as feedstock to a modified Fischer Tropsch process to produce a fuel, for example, jet fuel.

The combination of one anode 110, one anode compartment 110, one center compartment 120, one cathode 130, and one cathode compartment 130 is referred to herein as an “electrochemical unit” 200, although as discussed below, in some embodiments an anode compartment 110 and/or a cathode compartment 130 may be shared between adjacent electrochemical units 200. The compartments are provided inside “spacers”. The electrodes 110, 130 are housed in non-conductive “endblocks” 150, which may in turn be structurally supported by “endplates” 160. The endplates 160 at the ends are typically pulled together by multiple threaded rods and nuts (“tie-rods) to compress and seal the stack of endblocks 150, spacers and membranes 140 and counter the internal fluid pressure. In some designs an endblock 150 and an endplate 160 may be the same part, machined or molded from plastic, for example.

An electrode is typically fabricated from a solid or expanded titanium plate with a coating selected for the electrode solution and reaction, current density, cost, and operating lifetime. The coating may contain platinum or mixed metal oxides (MMO) such as iridium oxide, ruthenium oxide, or other metal oxides.

The assembly of endplates, endblocks, spacers, compartments, electrodes, membranes, and tie-rods are referred to as an “E-CEM module”. To increase the throughput from the E-CEM module illustrated in FIG. 1, the flow rate through the center compartment can be increased (residence time reduced) or the width of the center compartment can be increased.

The set of endplates, endblocks and tie-rods is referred to as a “closing mechanism” and is in effect an overhead requirement that does not function directly as part of the electrochemical process. It adds cost, weight, footprint, and maintenance requirements to a module.

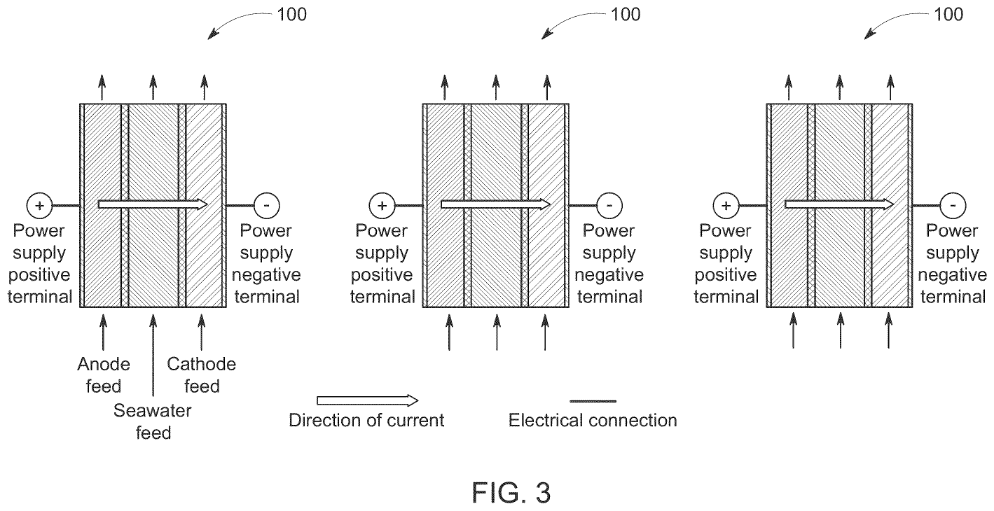

To further increase the production rate of an E-CEM system, multiple modules 100 can be installed and operated in parallel (see FIG. 3 for a simplified process schematic). Each module includes a set of closing mechanisms per center compartment. Each module may have its own DC power supply, or a single power supply may be used for multiple modules with the modules electrically connected in parallel.

It would be advantageous if a set of closing mechanisms could be used for multiple electrochemical units to provide an overall reduction in cost, weight, and footprint.

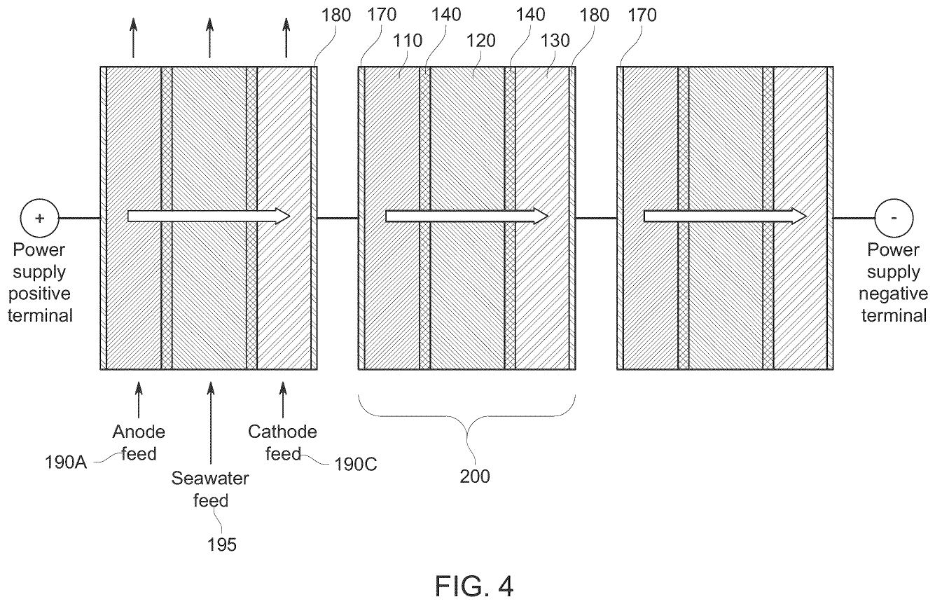

FIG. 4 shows a schematic of such an E-CEM system, with electrodes in multiple electrochemical units 200 connected back-to-back. Endplates and endblocks are omitted from FIG. 4 for clarity. A cathode 180 in an electrochemical unit 200 is connected to the anode 170 of the adjacent electrochemical unit 200. In one version, each electrode is housed in its own endblock. Compared to the system in FIG. 1, only one set of endplates and tie-rods is utilized for multiple center compartments, with consequent cost and weight reduction. The reduction in footprint may be limited, however, because of the space required for electrical connection between adjacent endblocks.

In a second version, the back-to-back electrodes may be housed in the same endblock. The design challenges then include method of connecting the electrodes inside the endblock and isolating the electrode streams.

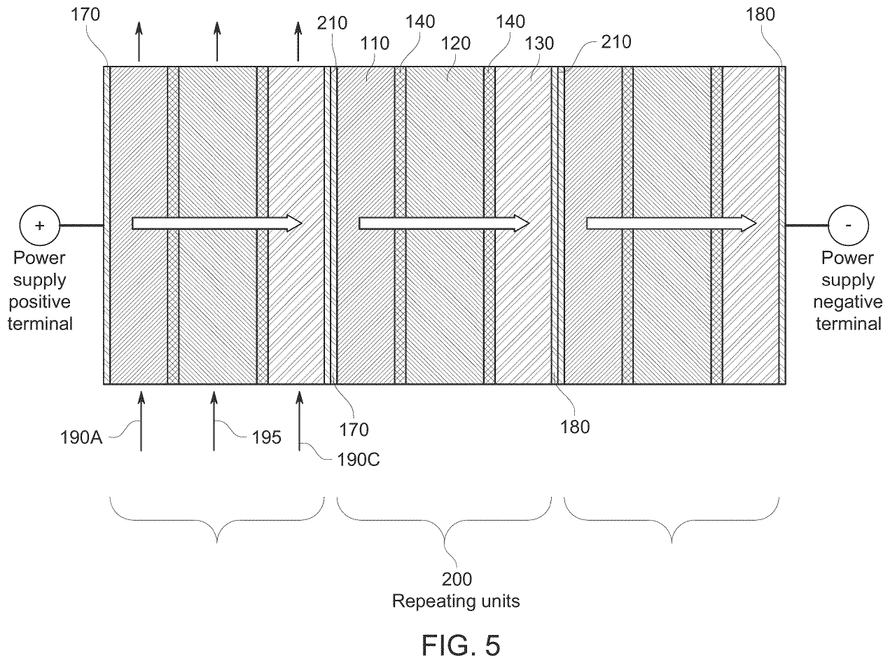

FIG. 5 shows a schematic of an E-CEM system with bipolar electrodes 210. Each bipolar electrode 210 is coated on both sides, with one side functioning as an anode 170 and the other as cathode 180. Compared to the system in FIG. 4, only one set of endplates and tie-rods (not shown) is utilized for multiple center compartments 120 and the number of endplates 160 is reduced from 2N to 2, where N=number of center compartments 120. The design challenges include isolation of the electrode streams on opposite sides of each bipolar electrode 210 and method of feeding the electrode streams, since the width of the electrode compartments 110, 130 may be on the order of 10 mm or less, for example, 3 mm or less to limit voltage drop per electrochemical unit 200.

The above referenced designs may be improved upon by utilizing porous electrodes separating the electrochemical units 200 in an E-CEM apparatus. As the term is used herein, porous electrodes include electrodes through with liquid electrolyte can flow and may include, for example, any of mesh electrodes, perforated sheet electrodes, sintered porous sheet electrodes, or other forms of liquid permeable electrodes.

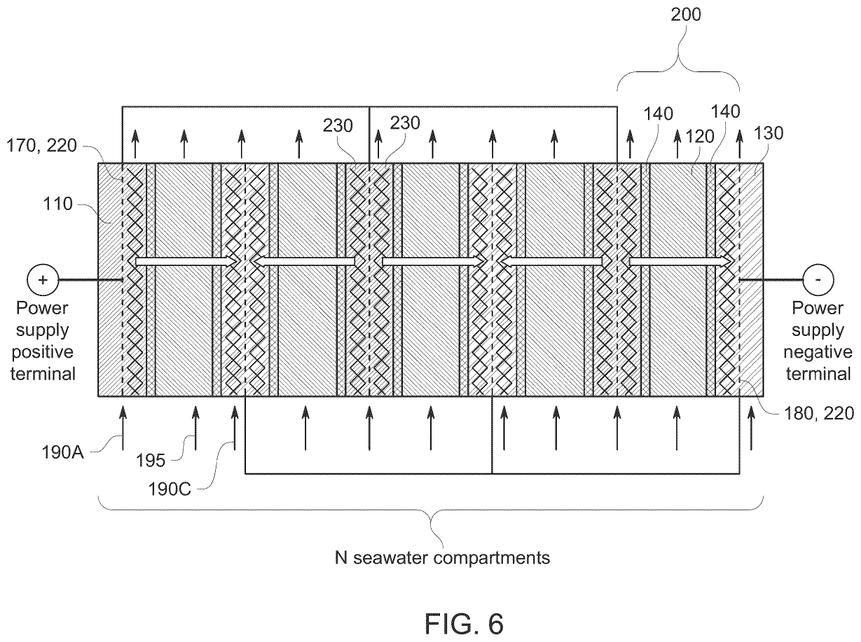

FIG. 6 shows a schematic of an example electrochemical system (an E-CEM system) with porous electrodes 220 and a plurality N of electrochemical units 200. Instead of a single electrochemical unit 200 as illustrated in FIG. 1, the electrochemical system of FIG. 6 includes at least one additional electrochemical unit 200 disposed between the first and second endplates (shown in FIG. 1). The at least one additional electrochemical unit 200 includes at least one additional porous anode 170 disposed in at least one additional anode compartment 110, at least one additional porous cathode 180 disposed in at least one additional cathode compartment 130, and at least one additional center compartment 120 disposed between and separated from the at least one additional anode compartment 110 and the at least one additional cathode compartment 130 by cation exchange membranes 140. In some embodiments the porous electrodes 220 are mesh electrodes including titanium electrode substrates fabricated from flattened expanded sheets with platinum, ruthenium oxide, iridium oxide, or mixed metal oxide (MMO) coatings on all surfaces contacting the electrode solutions 190A, 190C. In other embodiments the substrate can be fabricated from perforated sheets or sintered porous sheets, for example, which may also be coated with platinum, ruthenium oxide, iridium oxide, or mixed metal oxide (MMO). In some embodiments the anodes 170 and cathodes 180 may be formed of, or coated with, different materials. In some embodiments, the anodes 170 of each of the plurality of electrochemical units 200 may be formed of titanium coated with one of ruthenium oxide, iridium oxide, or a mixed metal oxide while the cathodes 180 of each of the plurality of electrochemical units 200 may be formed of titanium coated with platinum.

Each porous (e.g., mesh) electrode 220 is immersed in either the anode electrolyte solution 190A or the cathode electrolyte solution 190C. Isolation of the solutions 190A, 190C on both sides of the electrodes 220 is not necessary. The electrochemical system thus includes first and second endplates (for example, as shown in FIG. 1) and a plurality of electrochemical units 200 disposed between the first and second endplates. Each of the plurality of electrochemical units 200 includes a porous anode 170 disposed in an anode compartment, 110 a porous cathode 180 disposed in a cathode compartment 130, and a center compartment 120 disposed between and separated from the anode compartment 110 and the cathode compartment 120 by cation exchange membranes 140. At least one of the anode compartments 110 functions as an anode compartment 110 for adjacent electrochemical units 200. At least one of the cathode compartments 130 functions as a cathode compartment 130 for adjacent electrochemical units 200.

Inert porous screens 230 (only two shown in FIG. 6) are included in the anode compartments 110 and cathode compartments 130 and are placed between sides of the anodes 170 and cathodes 180 and adjacent CEMs 140 to support the membranes 140 from deforming into the spaces in the electrodes 220 and prevent direct electrical contact between the electrodes 220 and the membranes 140. The screens 230 can be fabricated from woven plastic strands or extruded plastic netting. The electrode solutions 190A, 190C can flow between the adjacent membranes 140 through the screens 230 and the pores in the electrodes 220. The porous structure of the electrodes 220 and/or the inert screens 230 can promote mixing in solution and enhance electrode reactions and ion transfer at the membranes 140.

The anodes 170 and cathodes 180 alternate in the module with current directions as shown in FIG. 6. The anodes 170 are electrically connected in parallel, as are the cathodes 180. Applying the electrical current across the anodes 170 and cathodes 180 of each of the plurality of electrochemical units 200 includes flowing current from each pair of two anodes 170 to the cathode 180 therebetween. Applying the electrical current across the anodes 170 and cathodes 180 of each of the plurality of electrochemical units 200 includes applying a voltage to each of the anodes 170 higher than the voltage applied to each of the cathodes 180 in parallel. Each current arrow represents a current which depends on the average current density at the electrodes 220 and the electrode area. The actual surface area of a mesh electrode 220 depends on the design of the mesh and is higher than the “superficial” area (the area based on the overall width and length of the electrode 220). The current density is typically expressed as the current divided by the superficial area. The maximum number of electrochemical units 200 in the module is limited by the maximum output current available from the power supply.

In some embodiments, the power supply electrically coupled to the anodes 170 and cathodes 180 of each of the plurality of electrochemical units 200 is configured to supply sufficient current across the anodes 170 and cathodes 180 of each of the plurality of electrochemical units 200 to acidify a feed stream in the center compartments 120. In some embodiments, the power supply is configured to supply sufficient current across the anodes 170 and cathodes 180 to acidify an aqueous solution in the center compartments 120 and generate hydrogen gas from catholyte 190C in the cathode compartments 130. In some embodiments, wherein seawater is used as feed 195 to the center compartments 120 the power supply is configured to supply sufficient current across the anodes 170 and cathodes 180 of each of the plurality of electrochemical units 200 to cause a pH of the seawater in the center compartments 120 to change to a level at which carbon dioxide is formed from bicarbonate in the seawater. Applying the electrical current across the anodes 170 and the cathodes 170 may include applying sufficient current across the anodes 170 and cathodes 180 to cause a pH of the seawater in the center compartment to change from about 8 to below 4.

In the system of FIG. 6 only one set of endplates and tie-rods is utilized for the N center compartments vs. N sets for the system with multiple modules as illustrated in FIG. 3. Overall, the cost, weight, and footprint of an E-CEM system with N center compartments is reduced when configured as illustrated in FIG. 6 versus as illustrated in FIG. 3.

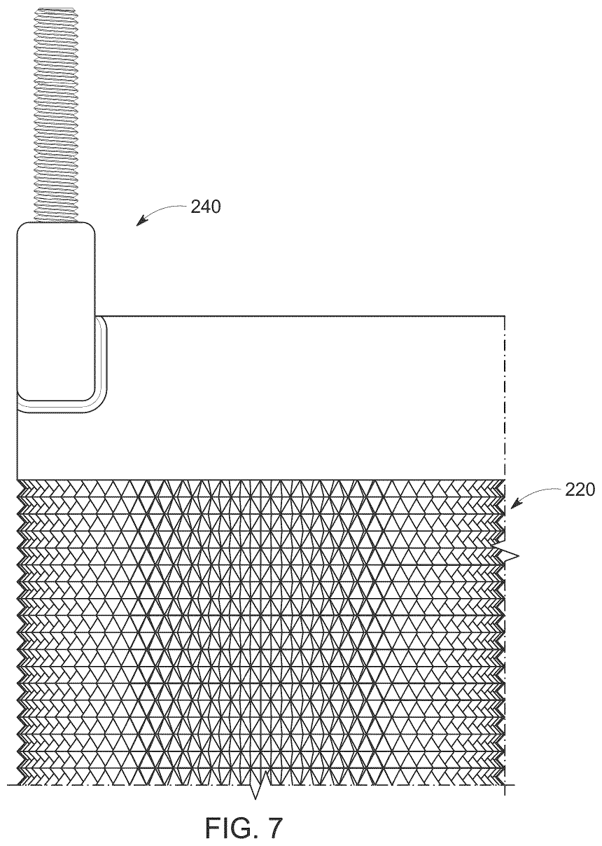

One design challenge is again the method of feeding the electrode electrolyte streams 190A, 190C since the width of the electrode compartments 110, 130 may be on the order of 10 mm or less, for example, 3 mm of less. Another challenge is design and sealing of the electrical connections to the electrodes. FIG. 7 shows a portion of an example of a mesh electrode 220 with an electrical connector 240 welded to one corner.

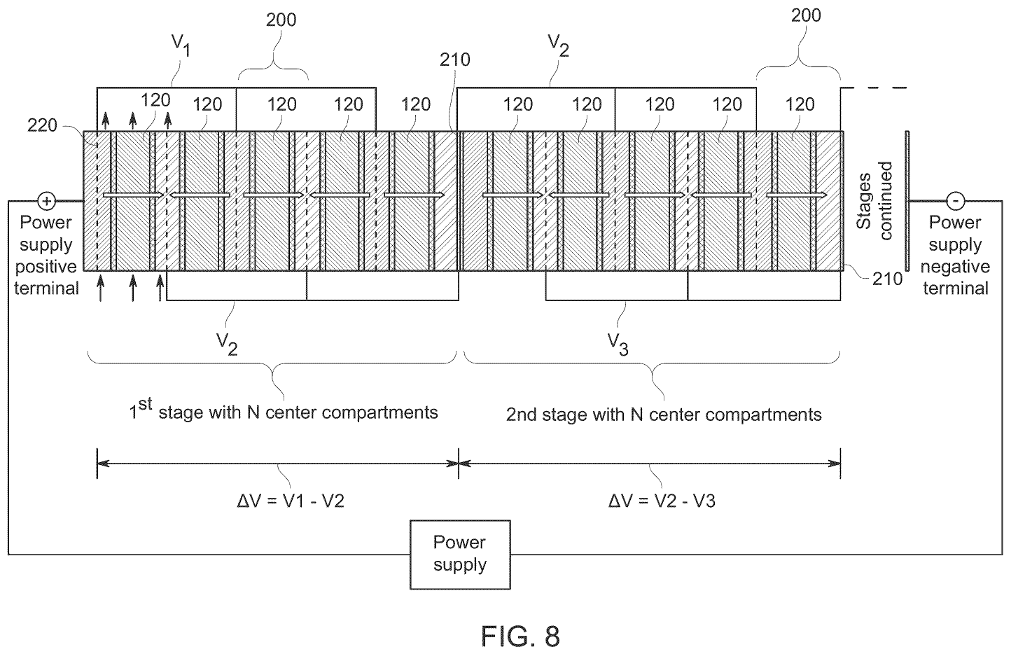

FIG. 8 shows a hybrid E-CEM system with porous (e.g., mesh) electrodes 220 and bipolar electrodes 210. At least one of the bipolar electrodes 210 is disposed between a first subset of the plurality of electrochemical units 200 and a second subset of the plurality of electrochemical units 200. At least one of the bipolar electrodes 210 separates an anode compartment 110 of the first subset of the plurality of electrochemical units 200 from a cathode compartment 130 of the second subset of the plurality of electrochemical units 200. In addition to applying electrical current across the anodes 170 and cathodes, electrical current may be applied to a bipolar electrode 210 disposed between a first subset of the plurality of electrochemical units 200 and a second subset of the plurality of electrochemical units 200. The electrochemical units in the module are divided into stages, each with N center compartments 120. The addition of solid bipolar electrodes 210 allows the total number of center compartments 220 to be further increased to a multiple of the number in FIG. 6. The design challenges are a combination of the challenges for the bipolar system shown in FIG. 5 and the mesh system in FIG. 6.

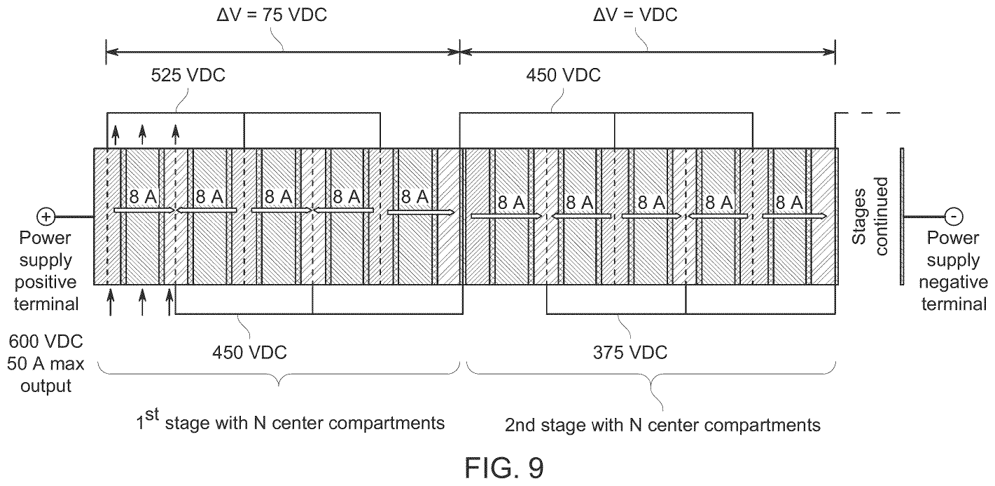

FIG. 9 shows a hypothetical example voltages and currents that may be utilized in the system of FIG. 8. Assuming that the current flowing through each center compartment 120 is 8 A and the voltage drop per stage (each stage including one electrochemical unit 200) is 75 VDC, the module can have up to 7 stages with 5 center compartments 120 per stage, assuming a power supply capable of providing 40 A at 525 VDC. The total number of center compartments is therefore 35, much higher than the number of center compartments in the example system of FIG. 5. The operating current and voltage are assumed to be lower than the maximum possible output from the DC power supply to allow some reserve during operation.

The systems shown in FIGS. 6 and 8 may be useful in applications other than acidification of seawater and recovery of CO₂ and H₂ gas for production of jet fuel. Both systems enable:
Acidification of the stream fed to the center compartments.
Production of an acidic stream with O₂ gas from the anode compartments. Cl₂ gas may also be present in the anode compartment effluents if Cl ions are present in the anode feed.
Production of a basic stream and H₂ gas from the cathode compartments.

Various operating parameters of the electrochemical systems disclosed herein may be controlled or adjusted by an associated control system or controller based on various parameters measured by various sensors located in different portions of the systems. The controller may be programmed or configured to regulate operating parameters such as flow rate of feed liquids through the anode compartments 110, center compartments 120, and cathode compartments 130 of systems disclosed herein as well as power (voltage or current) applied across electrodes 210, 220 of systems disclosed herein.

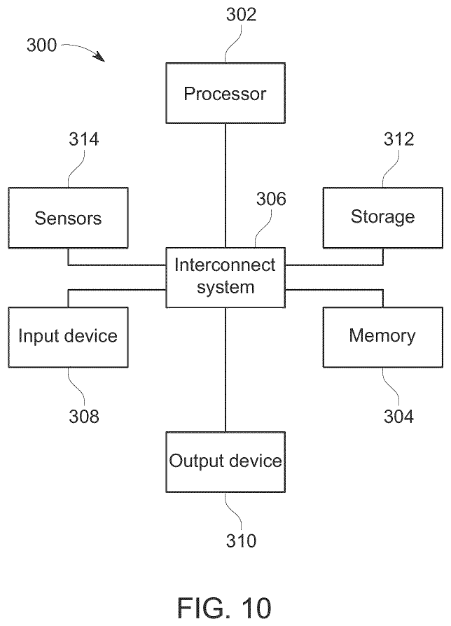

Various aspects of the controller may be implemented as specialized software executing in a general-purpose computer system 300 such as that shown in FIG. 10. The computer system 300 may include a processor 302 connected to one or more memory devices 304, such as a disk drive, solid state memory, or other device for storing data. Memory 304 is typically used for storing programs and data during operation of the computer system 300. Components of computer system 300 may be coupled by an interconnection mechanism 306, which may include one or more busses (e.g., between components that are integrated within a same machine) and/or a network (e.g., between components that reside on separate discrete machines). The interconnection mechanism 306 enables communications (e.g., data, instructions) to be exchanged between system components of system 300. Computer system 300 also includes one or more input devices 308, for example, a keyboard, mouse, trackball, microphone, touch screen, and one or more output devices 310, for example, a printing device, display screen, and/or speaker.

The output may also include analog or digital outputs to one or more power supplies to control the voltage or the current output from the power supplies.

The output devices 310 may also comprise valves, pumps, or switches which may be utilized to regulate or maintain flows of the various fluid streams of systems as disclosed herein. One or more sensors 314 may also provide input to the computer system 300. These sensors may include, for example, pressure sensors, chemical concentration sensors, temperature sensors, pH sensors, flow rate sensors, voltage sensors, current sensors, or sensors for any other parameters of interest to the systems disclosed herein. These sensors may be located in any portion of the system where they would be useful. In addition, computer system 300 may contain one or more interfaces (not shown) that connect computer system 300 to a communication network in addition or as an alternative to the interconnection mechanism 306.

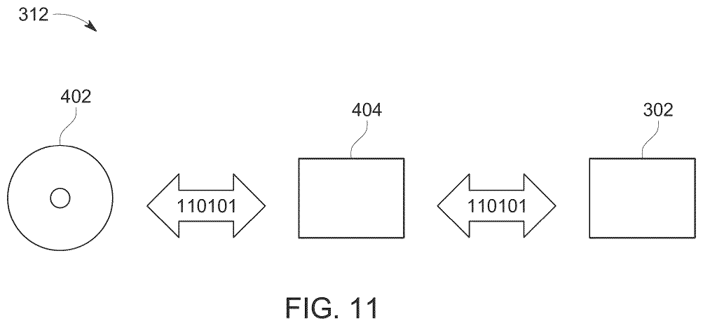

The storage system 312, shown in greater detail in FIG. 11, typically includes a computer readable and writeable nonvolatile recording medium 402 in which signals are stored that define a program to be executed by the processor 302 or information to be processed by the program. The medium may include, for example, a disk or flash memory. Typically, in operation, the processor causes data to be read from the nonvolatile recording medium 402 into another memory 404 that allows for faster access to the information by the processor than does the medium 402. This memory 404 is typically a volatile, random access memory such as a dynamic random access memory (DRAM) or static memory (SRAM). It may be located in storage system 312, as shown, or in memory system 304. The processor 302 generally manipulates the data within the integrated circuit memory 404 and then copies the data to the medium 402 after processing is completed. A variety of mechanisms are known for managing data movement between the medium 402 and the integrated circuit memory element 404, and aspects and embodiments disclosed herein are not limited thereto. Aspects and embodiments disclosed herein are not limited to a particular memory system 304 or storage system 312.

The computer system may include specially-programmed, special-purpose hardware, for example, an application-specific integrated circuit (ASIC). Aspects and embodiments disclosed herein may be implemented in software, hardware or firmware, or any combination thereof. Further, such methods, acts, systems, system elements and components thereof may be implemented as part of the computer system described above or as an independent component.

Although computer system 300 is shown by way of example as one type of computer system upon which various aspects and embodiments disclosed herein may be practiced, it should be appreciated that aspects and embodiments disclosed herein are not limited to being implemented on the computer system as shown in FIG. 10. Various aspects and embodiments disclosed herein may be practiced on one or more computers having a different architecture or components than shown in FIG. 10.

Computer system 300 may be a general-purpose computer system that is programmable using a high-level computer programming language. Computer system 300 may be also implemented using specially programmed, special purpose hardware. In computer system 300, processor 302 is typically a commercially available processor such as the well-known Core™ class processors available from the Intel Corporation. Many other processors are available, including programmable logic controllers. Such a processor usually executes an operating system which may be, for example, the Windows 10, or Windows 11 operating system available from the Microsoft Corporation, the MAC OS System X available from Apple Computer, the Solaris Operating System available from Sun Microsystems, or UNIX available from various sources. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that aspects and embodiments disclosed herein are not limited to a specific programming language or computer system. Further, it should be appreciated that other appropriate programming languages and other appropriate computer systems could also be used.

One or more portions of the computer system may be distributed across one or more computer systems (not shown) coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects and embodiments disclosed herein may be performed on a client-server system that includes components distributed among one or more server systems that perform various functions according to various aspects and embodiments disclosed herein. These components may be executable, intermediate (e.g., IL) or interpreted (e.g., Java) code which communicate over a communication network (e.g., the Internet) using a communication protocol (e.g., TCP/IP). In some embodiments one or more components of the computer system 300 may communicate with one or more other components over a wireless network, including, for example, a cellular telephone network.

It should be appreciated that the aspects and embodiments disclosed herein are not limited to executing on any particular system or group of systems. Also, it should be appreciated that the aspects and embodiments disclosed herein are not limited to any particular distributed architecture, network, or communication protocol. Various aspects and embodiments disclosed herein may be programmed using an object-oriented programming language, such as SmallTalk, Java, C++, Ada, or C#(C-Sharp). Other object-oriented programming languages may also be used. Alternatively, functional, scripting, and/or logical programming languages may be used, for example, ladder logic. Various aspects and embodiments disclosed herein may be implemented in a non-programmed environment (e.g., documents created in HTML, XML or other format that, when viewed in a window of a browser program, render aspects of a graphical-user interface (GUI) or perform other functions). Various aspects and embodiments disclosed herein may be implemented as programmed or non-programmed elements, or any combination thereof.

### EXAMPLE

The function and advantages of these and other embodiments can be better understood from the following example. This example is intended to be illustrative in nature and is not considered to be limiting the scope of the invention.

Two lab-scale E-CEM modules with different electrodes were operated under the same seawater residence time and current density. The first module, labeled “7A”, had a single seawater compartment and electrodes comprising solid titanium plates coated with mixed metal oxides (MMO). The second module, labeled “7B”, had five seawater compartments and electrodes comprising expanded titanium mesh, also coated with MMO.

The configurations of electrodes, membranes, and electrode and seawater compartments are illustrated by FIG. 1 for module 7A and FIG. 6 for module 7B.

Data was collected by a NOVUS LogBox data acquisition system. Continuous flow probes (InPro 4260i/SG/120 by Mettler Toledo) were used to measure the pH of the seawater and cathode effluents. The DC power supplies were operated in constant current mode, where the output voltage is adjusted automatically to maintain the output current at a set value.

The feed to the seawater compartments was a synthetic seawater with conductivity of approximately 49,000 μS/cm and pH of approximately 8, prepared with Instant Ocean® salt mix. The feed to the electrode compartments was a Na₂SO₄ solution with conductivity of approximately 250 μS/cm.

The key variables of residence time and current density were as follows:

Residence time in seawater compartment

| | Number of | Volume per | Total volume | Total | Resi- |
|---|---|---|---|---|---|
| | seawater | seawater | in seawater | seawater | dence |
| | compart- | compartment | compartments | flow rate | time |
| Module | ments | cm³ | cm³ | L/min | sec |
| 7A | 1 | 64.5 | 64.5 | 0.4 | 9.7 |
| 7B | 5 | 130.0 | 650.1 | 4 | 9.8 |

Current density

| | Active | Total | Current per | |
|---|---|---|---|---|
| | electrode | applied | seawater | Current |
| | area | current | compartment | density |
| Module | cm² | A | A | A/m² |
| 7A | 57.79 | 2.30 | 2.3 | 398 |
| 7B | 102.4 | 20.0 | 4.0 | 391 |

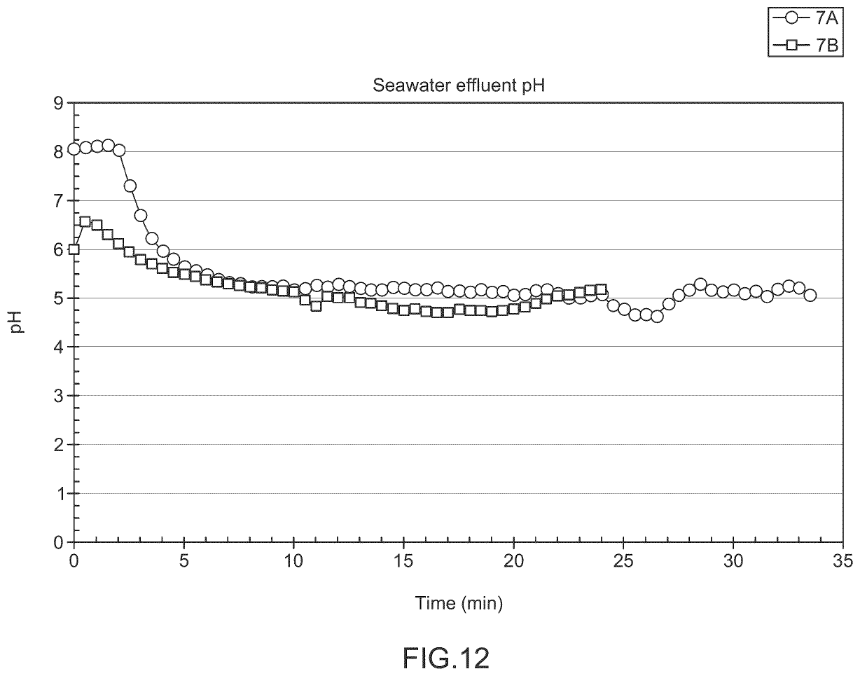

FIG. 12 shows that both modules reached a steady state pH of approximately 5 in the seawater effluent in about 10 minutes. Based on equilibrium calculation (see FIG. 2), approximately 93% of the HCO₃⁻ in the feed seawater was converted to H₂CO₃.

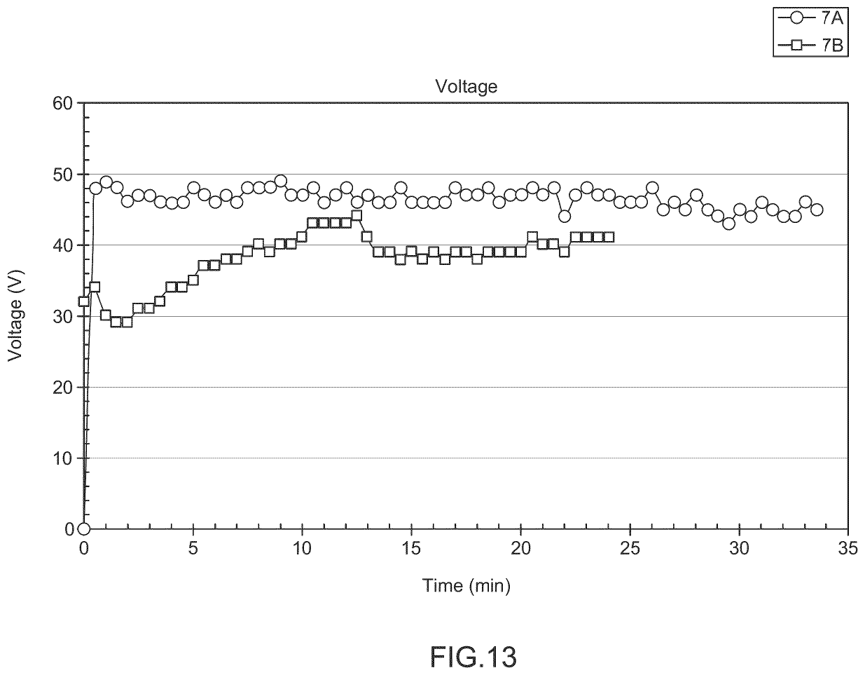

FIG. 13 shows the voltage applied to the modules to maintain the set current. The voltage for module 7A is higher than module 7B.

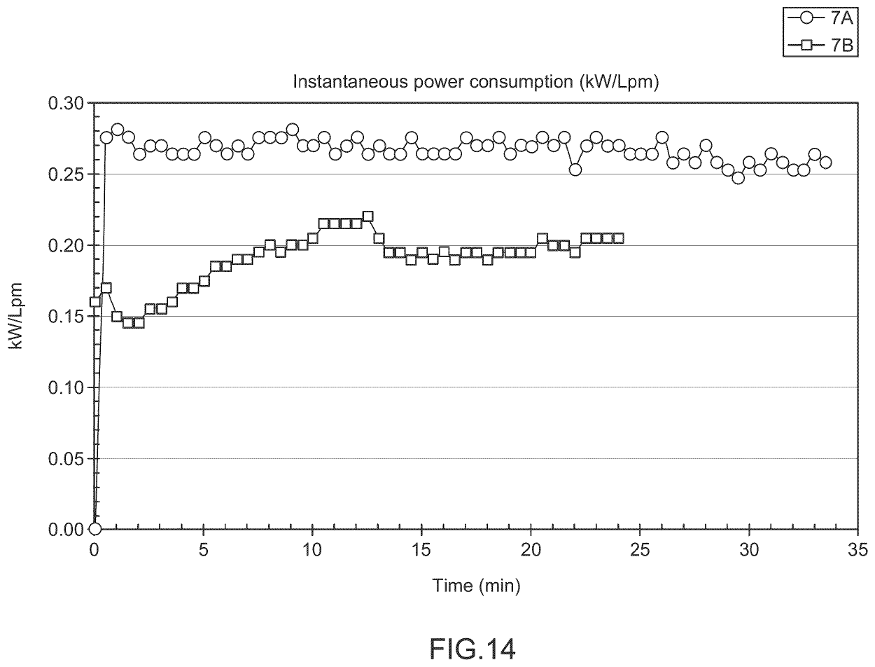

FIG. 14 shows the instantaneous power requirement per unit flow rate as kW/Lpm. Module 7A required over 25% more power than module 7B to achieve the steady state pH of approximately 5.

The tests showed that a module with mesh electrodes and multiple seawater compartments operated at least as well as a module of the original E-CEM design with solid electrodes and single seawater compartment. The reduction in energy requirement of 20% or more may be of advantage in applications for removing CO₂ from seawater.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Reference to features of the disclosed systems and methods in the plural also encompasses systems and methods including such features in the singular and reference to features of the disclosed systems and methods in the singular also encompasses systems and methods including such features in the plural.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. An electrochemical system comprising:
first and second endplates; and
a plurality of electrochemical units disposed between the first and second endplates, each of the plurality of electrochemical units including:
a porous anode disposed in an anode compartment;
a porous cathode disposed in a cathode compartment; and
a center compartment disposed between and separated from the anode compartment and the cathode compartment by cation exchange membranes.

2. The electrochemical system of claim 1, wherein at least one anode compartment functions as an anode compartment for adjacent electrochemical units.

3. The electrochemical system of claim 1, wherein at least one cathode compartment functions as a cathode compartment for adjacent electrochemical units.

4. The electrochemical system of claim 1, wherein the porous anode of each of the plurality of electrochemical units is formed of titanium coated with one of ruthenium oxide, iridium oxide, or a mixed metal oxide.

5. The electrochemical system of claim 1, wherein the porous cathode of each of the plurality of electrochemical units is formed of titanium coated with platinum.

6. The electrochemical system of claim 1, further comprising an electrical power supply, the porous anodes of each of the plurality of electrochemical units electrically coupled in parallel to the power supply, the porous cathodes of each of the plurality of electrochemical units electrically coupled in parallel to the power supply.

7. The electrochemical system of claim 1, wherein the porous anodes of each of the plurality of electrochemical units are one of mesh electrodes, perforated sheets, or sintered porous sheets.

8. The electrochemical system of claim 1, wherein the porous cathodes of each of the plurality of electrochemical units are one of mesh electrodes, perforated sheets, or sintered porous sheets.

9. The electrochemical system of claim 1, wherein the anode compartments further comprise inert porous screens.

10. The electrochemical system of claim 9, wherein the inert porous screens are disposed between the anodes and adjacent cation exchange membranes.

11. The electrochemical system of claim 1, wherein the cathode compartments further comprise inert porous screens.

12. The electrochemical system of claim 11, wherein the inert porous screens are disposed between the cathodes and adjacent cation exchange membranes.

13. The electrochemical system of claim 1, further comprising a source of electrolyte solution fluidly connectable to the anode compartment and the cathode compartment of each of the plurality of electrochemical units.

14. The electrochemical system of claim 1, further comprising a bipolar electrode.

15. The electrochemical system of claim 14, wherein the bipolar electrode is disposed between a first subset of the plurality of electrochemical units and a second subset of the plurality of electrochemical units.

16. The electrochemical system of claim 15, wherein the bipolar electrode separates an anode compartment of the first subset of the plurality of electrochemical units from a cathode compartment of the second subset of the plurality of electrochemical units.

17. The electrochemical system of claim 1, wherein the anode and cathode compartments have widths of about 10 mm or less.

18. The electrochemical system of claim 1, further comprising a power supply electrically coupled to the anodes and cathodes of each of the plurality of electrochemical units and configured to supply sufficient current across the anodes and cathodes of each of the plurality of electrochemical units to acidify a feed stream in the center compartments.

19. The electrochemical system of claim 1, further comprising a source of seawater fluidly connectable to the center compartments of each of the plurality of electrochemical units.

20. The electrochemical system of claim 19, further comprising a power supply electrically coupled to the anodes and cathodes of each of the plurality of electrochemical units and configured to supply sufficient current across the anodes and cathodes of each of the plurality of electrochemical units to cause a pH of the seawater in the center compartment to change to a level at which carbon dioxide is formed from bicarbonate in the seawater.

21.-37. (canceled)
